(19) United States
(12) Patent Application Publication
(10) Pub. No.: US 2024/0386552 A1
(43) Pub. Date: Nov. 21, 2024

(54) METHOD OF QUANTIFYING LUNG DISEASE

(71) Applicant: Vida Diagnostics, Inc., Coralville, IA (US)

(72) Inventors: Samuel W. Peterson, Topanga, CA (US); John D. Newell, Port Townsend, WA (US)

(21) Appl. No.: 18/666,070

(22) Filed: May 16, 2024

Related U.S. Application Data
(60) Provisional application No. 63/503,118, filed on May 18, 2023.
(60) Provisional application No. 63/595,138, filed on Nov. 1, 2023.

(51) Int. Cl. G06T 7/00; A61B 6/50; G06T 7/40

(52) U.S. Cl. CPC G06T 7/0012; G06T 7/40; A61B 6/50; G06T 2207/10081; G06T 2207/30061

(57) ABSTRACT

A method of using computed tomographic (CT) image data to provide a quantitative assessment of various classes of lung disease, including for example, interstitial lung abnormalities associated with interstitial lung disease (ILD) represented as a mass contribution, or as a percentage contribution by mass. Mass fractions of emphysema due to COPD are also addressed along with the special case of addressing air trapping mass fraction due to ILA/ILD, asthma, COPD, BOS.

### CROSS-REFERENCE TO RELATED APPLICATIONS

This application claims priority to U.S. Provisional Patent Application No. 63/503,118, filed May 18, 2023, and to U.S. Provisional Patent Application No. 63/595,138, filed Nov. 1, 2023, the contents of both which are incorporated herein by reference in their respective entireties.

### TECHNICAL FIELD

This disclosure is generally directed to the use of medical imaging data to assess lung disease. More particularly, this disclosure describes a method of using computed tomographic (CT) image data to provide a quantitative assessment of normal and diseased lung tissue, including interstitial lung abnormalities (ILA), changes in lung tissue due to emphysema, and air trapping, represented as a total mass, or as a percentage by mass. In some embodiments, the methods described herein provide a mass contribution, or a percentage contribution by mass, according to a tissue characterization scheme, such as by texture type, or by other methods of classifying disease and/or the absence thereof.

### BACKGROUND

The classification and quantification of lung disease, for example ILA features due to interstitial lung disease (ILD), can be challenging. When diagnosing or assessing lung disease, it is typically desirable to quantify aspects of one or more of those tissues and/or airspaces in the lungs based on images taken at one or more instants in time, for example, using computed tomography (CT). Some quantifications assume that scans for acquiring the analyzed data (e.g., CT scan data) have been acquired at full inspiration or full expiration, for example.

A current technique involves quantitative CT imaging that provides an assessment in terms of a volumetric percentage of a given lung region or volume. Various disease effects can impact the lung tissue in a way that gives it a distinctive appearance in volumetric scans of the lungs, such as grayscale lung CT images. The distinctive appearance of a portion of lung tissue may lend itself to labelling according to a classification scheme, for example, by texture type. For example, the texture of the lung parenchyma may be altered by diseases that may decrease or increase lung density. A current quantitative CT imaging (“qCT”) standard provides a percentage value for each texture type by volume (or as an absolute volume, e.g., measured in milliliters for example). For each texture type, the percent of that texture type in a given lung volume is determined by: (100× texture volume (mL)/region volume (mL)), where the texture may be selected from a list including, e.g., ground glass, reticulation, honeycombing, consolidation, emphysema, or normal lung tissue. The lung volume of interest (or region of interest, “ROI”) may be defined as desired, for example, the ROI could be defined as both lungs, just the left lung, just the right lung, one or more lobes of one or both lungs, sub-lobes, etc.

In addition to texture type, other classification schemes for characterizing and/or quantifying lung disease may also be employed. For example, scanned lung tissue could be characterized in a classification scheme as being either normal, air trapping, or emphysema, as described in U.S. Pat. No. 9,760,989, relevant portions of which are hereby incorporated by reference. Other examples of classification schemes for characterizing and/or quantifying lung disease may include, but are not limited to, criteria such as vessel content, high attenuating voxels (e.g., voxels whose Hounsfield Unit or “HU” value is in the range from −700 HU to −250 HU), expiratory air trapping (e.g., voxels whose HU value on expiration CT is less than −856 HU), or by employing criteria based on biomechanical properties such as elasticity (e.g., normal elasticity vs. impaired elasticity), or by classifying according to changes in density between expiration and inspiration, such as classifying “functional” lung (e.g., voxels whose HU value on inspiration is within a range of about −950 HU to about −600 HU) as several possible examples.

However, various issues can arise that may affect the accuracy or validity of such results. Variations in patient breath-hold (e.g., less than full lung capacity) during testing can negatively affect the result. The reason for this is that the air volume changes with the degree of inspiration or expiration but the tissue volume or tissue mass is conserved across the different lung volumes. For example, it is sometimes assumed that the scan was acquired at full inspiration (e.g., at total lung capacity, or TLC). However, a less than full inspiration can result in an apparent increase in the density of healthy lung tissue, which could then be mistaken for a texture pattern or some other imaging pattern that is associated with diseased lung tissue (e.g., ground glass opacities from acute pneumonia). For example, a diminished total lung capacity (TLC) breath-hold can increase the mis-detection of certain textures (e.g., ground glass), while also decreasing the lung or relevant region volume. Additionally, certain textures (e.g., ground glass and reticulation) may be treated equally using such measurement techniques despite variations in severity and/or concentration.

Thus, there exists a need for improved methods and systems for quantifying lung tissue that addresses and/or resolves the above-mentioned shortcomings in current techniques.

### SUMMARY

Certain embodiments of this disclosure are described herein with reference to illustrative embodiments.

Some embodiments of this disclosure include methods of characterizing lung disease, such as ILD, emphysema, and air trapping in residual volume (RV) scans, in terms of a percentage by mass or a total mass for one or more selections within a given classification/category (e.g., by texture type).

Some embodiments of this disclosure include methods to characterize lung disease according to texture type in terms of a total mass or a percentage by mass of a texture type in a given region of interest (“ROI”).

Some embodiments of this disclosure include methods to characterize lung disease according to a selection within a classification scheme in terms of a percentage by mass or a total mass in a given ROI.

Some embodiments of this disclosure include methods to characterize lung disease features (such as ILA due to ILD) according to texture in terms of a weighted percentage by mass in a given ROI of a patient's lungs using CT-based determinations of mass in Hounsfield units.

### BRIEF DESCRIPTION OF DRAWINGS

The following drawings are illustrative of particular embodiments of this disclosure and therefore do not limit the scope of this disclosure. The drawings are not necessarily to scale (unless so stated) and are intended to accompany the explanations in the following detailed description. Embodiments of this disclosure will hereinafter be described in conjunction with the appended drawings, wherein like numerals denote like elements.

FIG. 1 is a flowchart showing exemplary steps of acquiring and/or processing volumetric imaging data according to certain embodiments of this disclosure;

FIG. 2 is a flowchart showing exemplary steps of a method of characterizing lung tissue in terms of a total mass and/or a percentage by mass for each of a given texture type or class according to certain embodiments of this disclosure;

FIG. 3A illustrates examples of various disease-relevant texture types;

FIG. 3B illustrates additional examples of various disease-relevant texture types;

FIG. 4 is a flowchart showing exemplary steps of a method of characterizing lung tissue in terms of a total mass and/or a percentage by mass for each of a defined set of classification labels or classification types, according to certain embodiments of this disclosure;

FIG. 5 is a description of a classification scheme for characterizing lung tissue as being either normal, air trapping, or emphysema;

FIG. 6 shows optional or additional details for applying or implementing the disease probability measurement (“DPM”) classification scheme of FIG. 5; and

FIG. 7 is a description and certain details of a biomechanical classification scheme for characterizing lung tissue as being normal, impaired, or at risk.

### DETAILED DESCRIPTION

This disclosure describes one or more methods of providing quantitative assessments of lung tissue composition according to a classification or type (such as texture type, for example) in terms of a percentage by mass. The methods described herein may improve the consistency and therefore the clinical relevance of such assessments and thereby enable better clinical decision making.

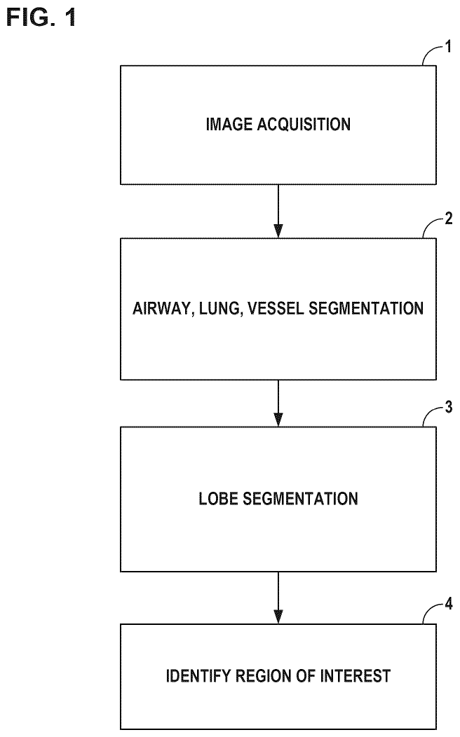

FIG. 1 is a flowchart showing exemplary initial steps of acquiring volumetric imaging data according to certain embodiments of this disclosure. At step 1, volumetric radiological images or imaging data of a patient are transmitted to the pulmonary imaging system. For example, a method according to certain implementations may include receiving volumetric pulmonary scan data representative of a patient's pulmonary structure. Alternatively, the volumetric radiological images or imaging data may already be stored within the memory of the system and may be accessed by a processor. The volumetric radiological images or imaging data may include CT scanned images, for example, from which a series of two-dimensional planar images can be produced in multiple planes, for example. Each image in the series of the multi-dimensional volumetric images provided by CT and MRI scans, for example, is a two-dimensional planar image that depicts the tissue present in a single plane or slice. These images are typically acquired in three orthogonal planes, which are referred to as the three orthogonal views and are typically identified as being axial, coronal and sagittal views.

At step 2 in FIG. 1, the lungs, airways, and/or blood vessels are segmented using the volumetric image data acquired in step 1. For example, a method may include processing the received volumetric pulmonary scan data to identify one or more anatomical structures within the volumetric pulmonary scan data. The methods of performing lung, airway and vessel segmentation from the volumetric images or imaging data may be those employed by the Pulmonary Workstation of VIDA Diagnostics, Inc. (Coralville, Iowa) and as described in the following references, each of which is incorporated herein by reference in relevant part: U.S. Pub. 2007/0092864, entitled, “Treatment Planning Methods, Devices and Systems”; U.S. Pub. 2006/0030958, entitled, “Methods and Devices for Labeling and/or Matching”; Tschirren et al., “Intrathoracic airway trees: segmentation and airway morphology analysis from low-dose CT scans,” IEEE Trans Med Imaging. 2005 December; 24 (12):1529-39; Tschirren et al., “Matching and anatomical labeling of human airway tree,” IEEE Trans Med Imaging. 2005 December; 24 (12):1540-7; Tschirren, Juerg, “Segmentation, Anatomical Labeling, Branchpoint Matching, and Quantitative Analysis of Human Airway Trees in Volumetric CT Images,” Ph.D. Thesis, The University of Iowa, 2003; Tschirren, Juerg, Segmentation, Anatomical Labeling, Branchpoint Matching, and Quantitative Analysis of Human Airway Trees in Volumetric CT Images, Slides from Ph.D. defense, The University of Iowa, 2003; and Li, Kang, “Efficient Optimal Net Surface Detection for Image Segmentation—From Theory to Practice,” M.Sc. Thesis, The University of Iowa, 2003. Segmentation of the lungs, airways, and vessels results in identification of the lungs, airways, and vessels as distinct from the surrounding tissues, and separates the lungs, airways, and vessels into smaller distinct portions, which may be individually identified in accordance with standard pulmonary anatomy.

At step 3 in FIG. 1, lobar segmentation may optionally be performed. The segmentation of the lungs, airways, and vessels obtained in step 2 can be used to identify and delineate the lobes, again by applying standard pulmonary anatomy. For example, using the identified segments of the airway and/or vessel trees obtained in step 2, the lobes may be segmented and identified by extracting the portions of the airway tree corresponding to particular lobes based on known air way tree structures and connectivity information. The extracted lobar airway tree portions may be further divided into portions corresponding to sub-lobes, again based on known airway and/or vessel tree structure and connectivity information. In this way, the portions of the volumetric images corresponding to lobes and/or sub-lobes can be identified.

At step 4 in FIG. 1, the region (or regions) of interest (ROI) is/are identified. This may be an automatically performed step, or it may involve manual selection of the ROI by a user via a graphical software interface, for example. The ROI will define a specific set (e.g., a finite set) of voxels for performing the analysis.

The following example is described using one particular classification scheme—classification of tissue by texture type. However, it should be noted that the concepts described herein and the steps performed can be extended by analogy to other classification schemes for characterizing lung tissue, some of which are described in more detail below.

Typically, there are about 5-7 texture types or classes of interest for the purpose of quantifying normal and diseased lung tissue, including interstitial lung abnormalities due to interstitial lung disease. Commonly, these texture types may include ground glass, reticulation, emphysema, air trapping on RV scans (a measurement of the volume of air that remains in the lungs after maximal exhalation), honeycombing, consolidation, and normal lung tissue. In some cases, it may be desirable to establish one or more additional “hybrid” texture types comprising features from two or more categories. For example, a “ground glass with reticulation” texture type may be defined where there are features of both ground glass and reticulation. Additionally, emphysema may sometimes be categorized further into various sub-types based on distribution patterns (e.g., paraseptal emphysema, panlobular emphysema, and centrilobular emphysema). Other texture patterns might include traction bronchiectasis, mosaic attenuation, tree-in-bud, cysts, and small nodule patterns.

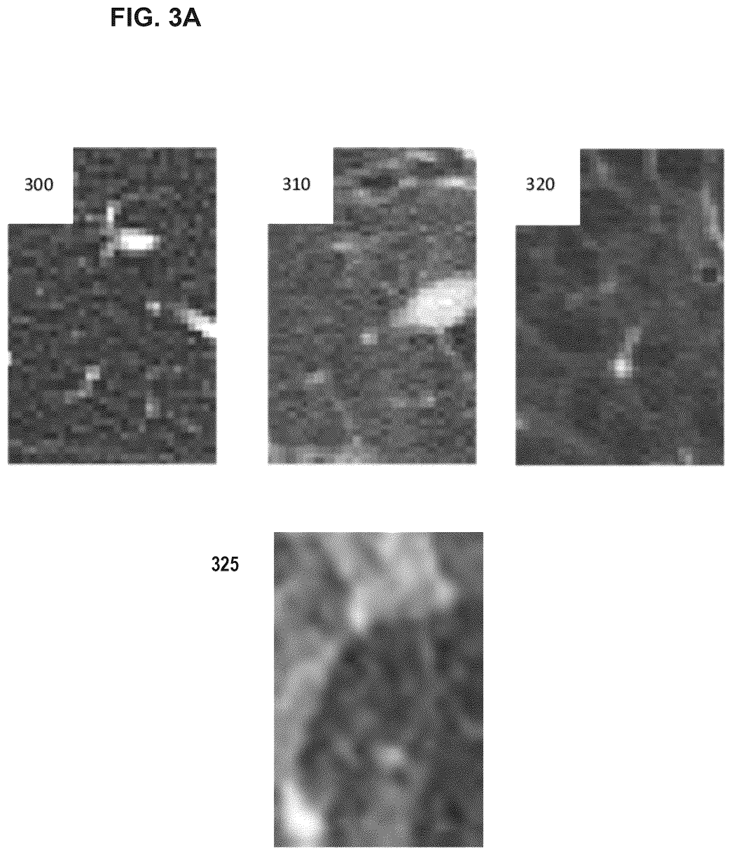

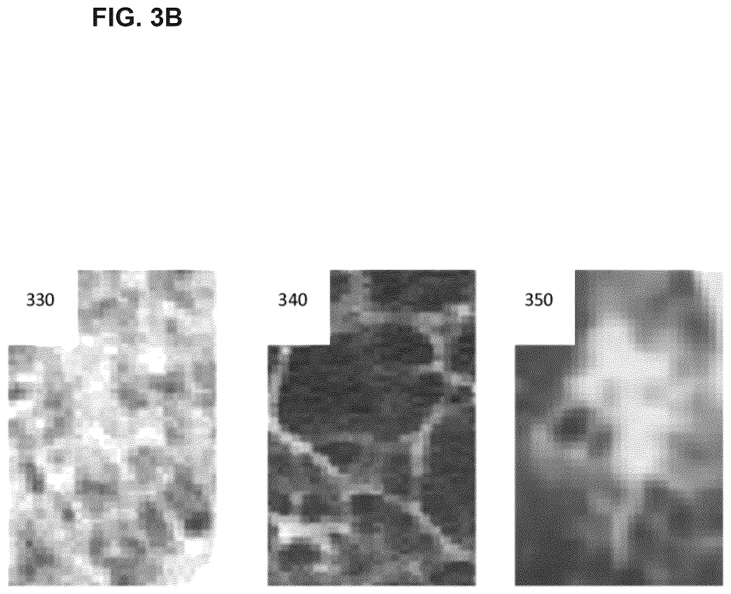

Some examples of the various disease-relevant texture types are shown in FIG. 3A and FIG. 3B. As shown, texture 300 may be representative of a normal lung parenchyma texture, texture 310 may be representative of a ground-glass texture, texture 320 may be representative of an emphysema texture, texture 325 may be representative of a texture corresponding to air trapping in a residual volume (RV) scan, texture 330 may be representative of a reticulation texture, texture 340 may be representative of a honeycombing texture, and texture 350 may be representative of a consolidation texture. Detecting and measuring the amount and distribution of various texture types, such as the texture types shown in FIG. 3A and FIG. 3B, throughout the lung can provide information for diagnosing, subtyping, and/or quantifying the progression of various pulmonary diseases. Embodiments of this disclosure include methods of detecting and measuring the amount and distribution of various texture types based on mass (and/or percentage contributions by mass); using mass (rather than volume, for example) as the basis for quantifying the amount and distribution of texture types may help avoid certain errors caused by invalid assumptions (e.g., assuming that a patient's lungs are at full inspiration or full expiration during a volume-based measurement).

In the series of two-dimensional images that make up the volumetric image data, a method according to some embodiments may include defining a volume region of interest (“ROI”). This ROI could be defined in a number of ways, as appropriate or as needed. For example, the ROI could be defined as both lungs, or either one of the lungs individually, or a lobe of a lung, or a sub-lobe, or a user-defined portion of a lung or lungs, for example. In a method according to some embodiments, a user may then assign a texture label or class to each voxel in the ROI according to its identified texture type. In some embodiments, the labeling of voxels according to texture type may be automated. For example, VIDA Diagnostics, Inc. (Coralville, Iowa) provides a fully-automated algorithm that assigns a pre-defined lung texture label to every voxel within the ROI of a given scan volume. In some embodiments, for example, assigning a texture label or class to each voxel in the ROI may comprise comparing the received volumetric pulmonary scan data to a database. In some embodiments of such a method, the received volumetric pulmonary scan data may be compared to a lung texture database. In some further embodiments of such a method, the comparison of volumetric pulmonary scan data to a lung texture database may be automated (e.g., by a software program or application) so that voxels within the volumetric pulmonary scan data are assigned a lung texture class (or label, or type) from a given set of lung texture types, for example. Such a data base could be used, for example, to train a machine learning algorithm to recognize the specific features of each set and assign texture labels to the data.

At this point, the technique may comprise a weighting process by which the contribution of each voxel in the ROI is weighted by the mass associated with that particular voxel. For example, with CT scanned images, the intensity of a given voxel is determined or measured (e.g., in terms of absorption and/or attenuation coefficients) in Hounsfield Units (HU), which may be used to calculate the mass of a particular voxel. The Hounsfield Units measurement is effectively a density measurement, which can be used to calculate the mass associated with a particular voxel. An equation for calculating the mass of a voxel based on the Hounsfield Units (HU) associated with that voxel is:

$$Mass = ( ( 10 \; 00 + HU ) / 10 \; 00 ) \times ( voxel volume , in {mm}^{3} ) in mg$$

It should be noted that there are variations to this equation (for example, sometimes a slightly different number, such as the number 1023, is used instead of the 1000 that appears in both the numerator and denominator of the above equation). More complex methods might use image samples taken from “pure air” (typically sampled from the trachea) and/or “pure blood” (typically sampled from the aorta) in order to compute the mass in a way that is scan-specific, for example.

Discrepancies caused by variations in breath-hold may manifest themselves and/or become even more significant as measurements are made over longer periods of time, for example when comparing a succession or series of measurements taken at various points in time to establish a trend (e.g., for the purpose of diagnosis). The errors that can arise may make it difficult to compare such results to each other, and/or to make sense of any trends in the data, which could lead to errors in clinical decision-making, for example. The mass weighting used in this technique may help avoid or minimize the effects of such errors that may be caused by volume variations in total lung capacity (TLC) breath-hold from test to test, as well as volume variations in RV type breath-hold scans. This is because tissue mass (unlike volume) remains mostly constant throughout the entire breath cycle; the tissue mass of a given ROI is roughly the same at full expiration as it is at full inspiration. That is, the mass of such tissue is conserved across the different lung volumes.

As noted above with respect to prior techniques, a diminished TLC breath-hold can increase the mis-detection of certain textures (e.g., ground glass), while also decreasing the total lung volume. In percentage terms, this could compound a potential error by both increasing the numerator and decreasing the denominator in a typical percent volume calculation. Additionally, certain textures (e.g., ground glass, reticulation, emphysema) may be treated equally despite variations in severity and/or concentration. This may be due to lung tissue associated with these textures retaining some ability to expand/contract; thus, a less-than-full breath-hold may result in over-detection and/or mis-characterization of ground glass or reticulation, for example, while an insufficient or non-maximal inspiratory breath-hold may result in under-detection and/or mis-characterization of emphysema, for example.

As noted above, the calculation of the mass of each voxel in the anatomical ROI is performed based on its Hounsfield Unit (HU) density. A formula for determining this voxel mass is:

$$Mass , in mg = [ ( 10 \; 00 + HU ) / 10 \; 00 ] \times ( voxel volume , in {mm}^{3} ) ,$$

where voxel volume is the volume constituted by a single image voxel in mm³. The sum of the mass of all voxels across the entire region of interest is Mass_{region}. As noted above, it should again be noted that there are possible variations to this equation (for example, sometimes employing a slightly different number, such as the number 1023, instead of the 1000 that appears in the above equation).

Next, the total mass contribution for each texture type is calculated (e.g., for each texture type or class, the mass of all voxels that were classified as that type is summed to form a total for that type). The sum of the mass of all voxels across the ROI corresponding to texture type “i” is thereby determined as Massᵢ.

The density of a tissue in a CT scanned image may be represented using the Hounsfield system. In the Hounsfield scale, water, for example, has a value of zero Hounsfield Units (0 HU). A tissue that is denser than water has a positive value in terms of HUs (e.g., +X HU). And a tissue that is less dense than water has a negative value in terms of HUs (e.g., −X HU). Hounsfield Units are dimensionless units, obtained from a linear transformation of attenuation coefficients. The formula for Hounsfield Units is:

$$HU = ( \frac{μMATERIAL - μWATER}{μWATER} ) \times 1000 ,$$

$$where$$

$$μ = CT linear attenuation coefficient$$

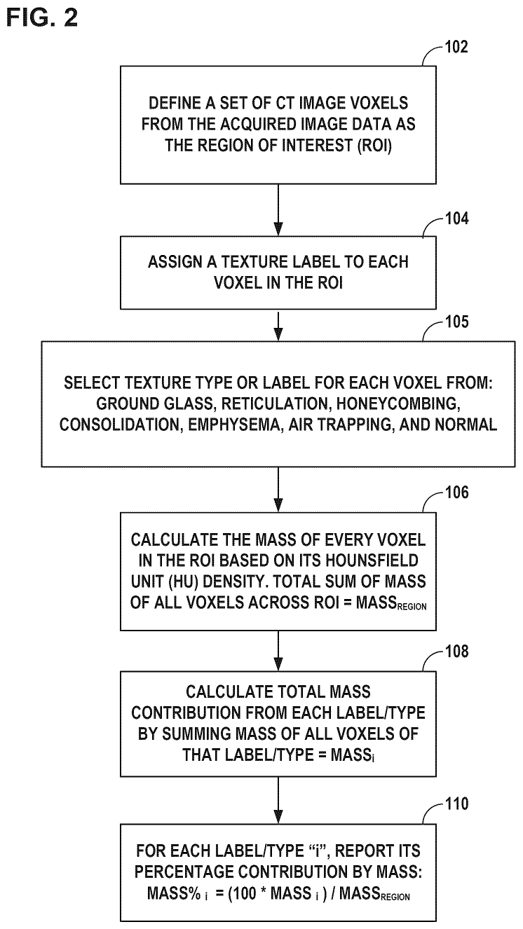

FIG. 2 is a flowchart further illustrating the above-described method of providing a quantitative assessment of various texture types or classes of interstitial lung disease (ILD) represented as a percentage by mass. The steps of the method are described in more detail below with reference to FIG. 2.

In step 102 in FIG. 2, define the overall set of CT image voxels comprising the region of interest (ROI).

In step 104 in FIG. 2, assign a texture label (e.g., texture type or texture class) to each voxel in the anatomical ROI based on texture type or texture class.

In step 105 in FIG. 2, each voxel in the ROI may be assigned a single texture type selected from a finite or predetermined list of texture types. The following exemplary list of texture types or classifications may be used to characterize each voxel of the ROI: “normal,” “ground glass,” “reticulation,” “honeycombing,” “consolidation,” “air trapping,” or “emphysema,” for example.

In step 106 in FIG. 2, calculate the mass of every voxel in the anatomical ROI based on its Hounsfield Unit (HU) density, e.g., using the formula for mass described above.

The total sum of the mass of all voxels (of all texture types) across the ROI=mass_{region}.

In step 108 in FIG. 2, calculate the total mass contribution of each texture type (or label or class) “i” by summing the mass of all voxels of that label/type in the ROI=massᵢ.

In step 110 in FIG. 2, for each texture type “i,” report its percentage contribution by mass as follows: mass %ᵢ=(100 * massᵢ)/mass_{region}.

Thus, in a given ROI for imaging data of a patient's lungs, a percentage by mass (e.g., a weighted percentage by mass) will be provided for each texture type within the ROI according to some embodiments of this disclosure. It should also be noted that the total mass for a given texture type may also be useful as a measure of disease extent (e.g., without necessarily determining the percentage contribution by mass for the given texture type). This may, for example, be true for certain diseases where the total mass of the lung decreases over time (such as in chronic obstructive pulmonary disease, or COPD); in such cases, monitoring for a change and/or tracking the amount of change in total mass for a given texture type over time may provide a good measure of disease extent and/or disease progression in such cases. This may be beneficial, for example, if computer processing resources and/or processing time are limited or costly. As one possible example of this, detecting a change in mass for a single texture type over time may provide clinically relevant information without the need to employ additional computer processing resources and/or time to compute the mass of all other texture types, or to compute percentage contributions based thereon.

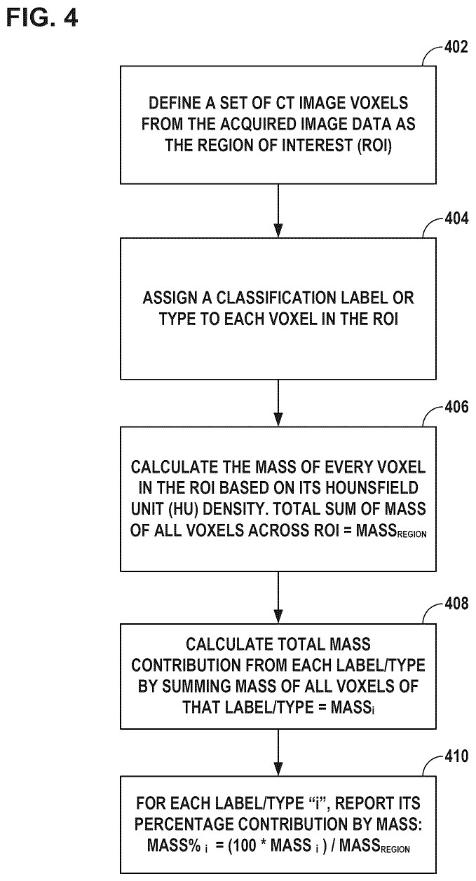

In FIG. 4, the technique described with reference to FIG. 2 has been “generalized” to apply to the use of other classification schemes (beyond the texture type classification scheme described with reference to FIG. 2). FIG. 4 is a flowchart showing exemplary steps of a method of characterizing lung tissue composition represented as a percentage by mass for each of a selected classification type, according to certain embodiments of this disclosure. Typically, there would be a defined or predetermined number of classification types within such a classification scheme. In the example of using texture types as the classification scheme, there may be as many as 7 or more classification types according to texture. In other classification schemes, such as the disease probability measurement (“DPM”) classification scheme or a classification scheme based on biomechanical properties of lung tissue, there may be only 3 or 4 classification types, or perhaps as few as 2, for example. These exemplary classification schemes will be described in more detail further below, although it is noted that there may be other classification schemes that could be employed according to the method described in FIG. 4, which may include some or all of the following steps:

In step 402 in FIG. 4, the overall set of CT image voxels comprising the region of interest (ROI) is defined from the acquired image data.

In step 404 in FIG. 4, a classification type or label (e.g., selected from a classification scheme comprising a list of classification types or labels) is assigned to each voxel in the anatomical ROI.

In step 406 in FIG. 4, the mass of every voxel in the anatomical ROI is calculated based on its Hounsfield Unit (HU) density, e.g., using the formula for mass described above. The total sum of the mass of all voxels (of all classification types or labels) across the ROI=mass_{region}.

In step 408 in FIG. 4, the total mass contribution of each classification type or label “i” is calculated by summing the mass of all voxels of that label/type in the ROI=massᵢ.

In step 410 in FIG. 4, for each classification label or type “i,” the percentage contribution by mass is calculated and reported as follows: mass %ᵢ=(100 * massᵢ)/mass_{region}.

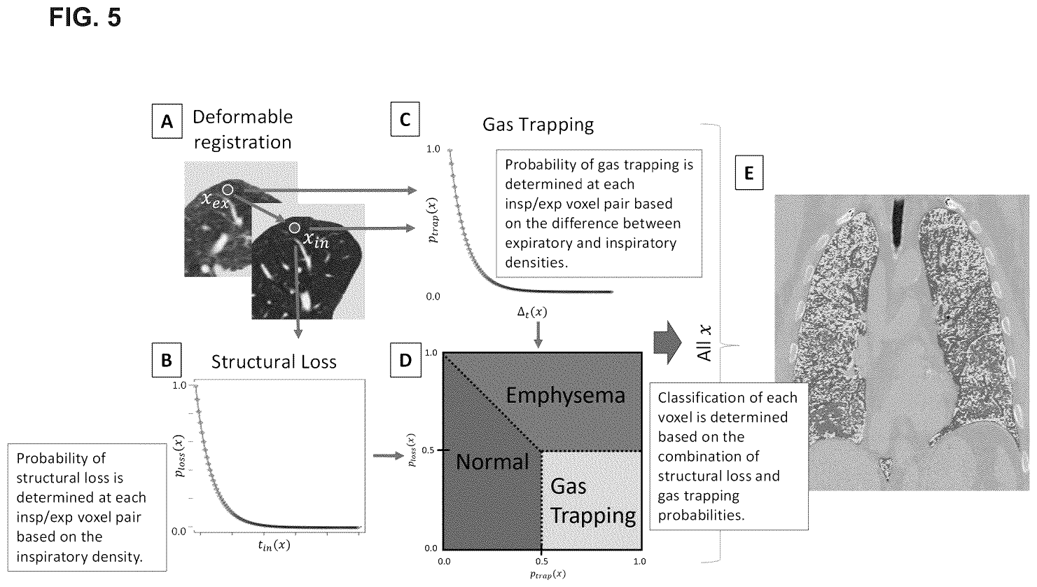

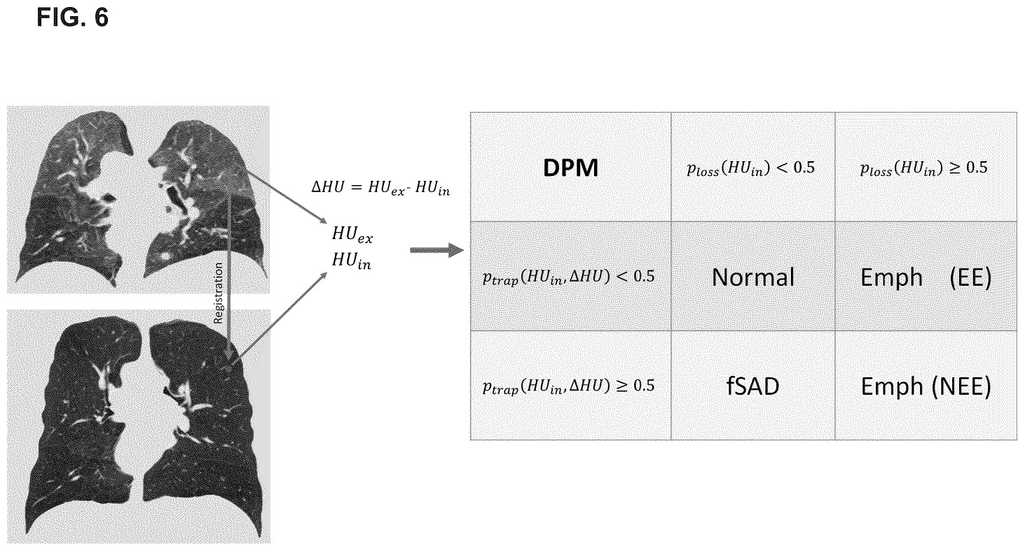

The method according to the flowchart of FIG. 4 may be employed using a classification scheme such as the disease probability measurement (or “DPM”) classification scheme described in U.S. Pat. No. 9,760,989. FIGS. 5 and 6 show certain aspects of classifying tissue according to the classification types of normal, air/gas trapping, or emphysema. Panel “E” of FIG. 5, for example, shows a scan of an ROI (the ROI in this example corresponding to lung tissue of the whole lung) in which each voxel of the scanned image has been classified into one of the three defined classification types of the DPM scheme (normal, air/gas trapping, or emphysema), and the remaining steps of the method of FIG. 4 can be applied to determine the percentage contribution by mass of each classification type. In FIG. 5, panel “E,” for example, each voxel is assigned to one of the three classifications, which may be represented using three distinct colors or shading to represent the three classifications of normal, air/gas trapping, and emphysema. Panel “D” of FIG. 5 shows exemplary criteria for assigning each voxel to the three classifications of the DPM scheme (e.g., using the probability of structural loss plotted against the probability of gas trapping, determined for each inspiration/expiration voxel pair).

FIG. 6 provides additional details of the DPM classification scheme, including determining a change in the HU level for a given voxel during inspiration vs. expiration and using that information to determine which classification type applies and/or to further refine the classification process. For example, the classification type may be based on vessel content, such as by defined ranges of HU values, or by categorizations of high attenuating voxels (e.g., voxels whose HU value is in the range from −700 HU to −250 HU), or by expiratory air trapping (e.g., voxels whose HU value on expiration is less than −856 HU), according to various embodiments.

In some cases, the likelihood of classification of tissue as emphysema may be determined solely based on the inspiratory voxel value (e.g., shown in the right column of the table in FIG. 6). For example, if that likelihood is >0.5, then the voxel is labeled “emphysema.” If that likelihood is <0.5, then one may look at the change in density of a voxel from inspiratory to expiratory to determine a probability of whether or not the voxel exhibits air trapping. If the voxel exhibits air trapping (but not emphysema), then the voxel is labeled as representing functional small airways disease (fSAD), as shown in the table of FIG. 6.

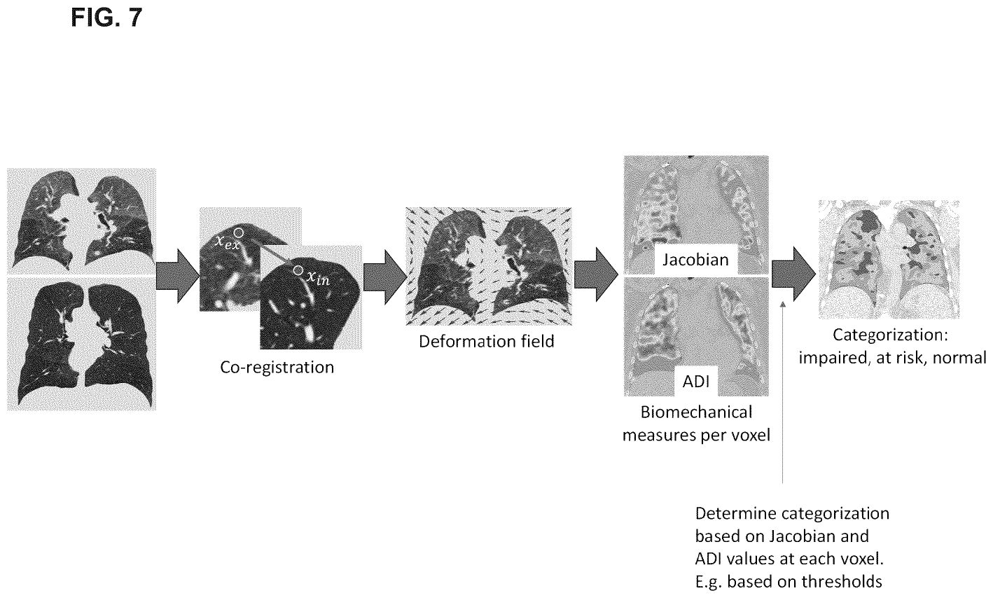

FIG. 7 illustrates an example of classifying tissue according to certain biomechanical properties, for example, to classify lung tissue into the classification types of normal, impaired, or at risk. The image at the far right of FIG. 7, for example, shows a scan of an ROI (corresponding to lung tissue) in which each voxel of the scanned image has been classified into one of the three defined biomechanical classification types (normal, impaired, or at risk), and the remaining steps of the method of FIG. 4 can then be applied to determine the total mass contribution or the percentage contribution by mass of each classification type.

FIG. 7 shows a process flow diagram of how various parameters and/or assessments of biomechanical properties may be used to determine which of the defined classification types will be assigned to each voxel. For example, in FIG. 7, the “Jacobian” analysis is based on the amount of volume change at a given voxel (e.g., between expiration and inspiration), whereas the “ADI” analysis may assess the degree or nature of the deformation irregularity at each voxel. The results of these analyses may be combined (generally as shown in FIG. 7) to determine a resulting classification type for each voxel in the ROI. It should be noted that, in some cases, the biomechanical classification scheme could be further simplified into two rather than three classification types; for example, criteria may be employed to classify tissue as either normal or impaired. In some embodiments, one could use a variety of biomechanical measures to assess whether a voxel is behaving like “normal” lung tissue versus abnormal/impaired lung tissue.

Thus, in a given ROI for imaging data of a patient's lungs, a percentage by mass (e.g., a weighted percentage by mass) may be provided for each classification or type within the ROI. It should also be noted that the total mass for a given classification type may also be useful as a measure of disease extent (e.g., without necessarily determining the percentage contribution by mass for the given classification type). This may, for example, be true for certain diseases where the total mass of the lung decreases over time (such as in chronic obstructive pulmonary disease, or COPD); tracking the change in total mass for a given classification type over time may provide a good measure of disease extent and/or disease progression in such cases.

In some situations, methods according to various embodiments of this disclosure may include the additional and/or optional step of aggregating the above-described texture-by-mass quantification methods to determine a total disease burden (also based on mass rather than volume). This may involve, for example, summing the mass contribution percentages of various textures to create a number of different composite scores. As possible examples, one might determine a percent quantitative lung fibrosis (“% QLF”) as being the sum of the % reticulation and % traction bronchiectasis (if defined and/or available) measures, again determined as a percentage by mass (rather than volume), as described above. Similarly, one could employ this technique to determine a percent quantitative ILD (“% QILD”) as being the sum of the % QLF, % ground glass, and % honeycombing measures, again determined as a percentage by mass (rather than volume), as described above. These exemplary measures may be represented in terms of the following formulas:

% QLF (quantitative lung fibrosis)=% reticulation+% traction bronchiectasis; and

% QILD=% QLF+% ground glass+% honeycombing.

Of course, other comparable scores may be defined as needed and/or as technology evolves in the future, and such methods are contemplated and deemed to be within the scope of this disclosure.

In the foregoing detailed description, inventive concepts have been described with reference to various illustrative embodiments. However, it may be appreciated that various modifications and changes can be made without departing from the scope of the invention.

What is claimed is:

1. A method of characterizing lung disease features in terms of mass for each of a given texture type within a region of interest, the method comprising:
acquiring volumetric imaging data comprising a plurality of voxels;
defining a region of interest within the volumetric imaging data;
assigning a texture type to each voxel within the region of interest, the texture type for a given voxel selected from a predetermined list of texture types;
calculating a mass for each voxel based on its Hounsfield Units attenuation coefficients; and
calculating a total mass of a first texture type within the region of interest by summing the total mass of all voxels of the first texture type within the region of interest.

2. The method of characterizing lung disease features of claim 1, further comprising:
monitoring the total mass of the first texture type within the region of interest over time.

3. The method of characterizing lung disease features of claim 1, further comprising:
calculating a total mass of all voxels within the region of interest by summing the mass of all voxels within the region of interest; and
determining a percent contribution by mass of the first texture type within the region of interest.

4. The method of characterizing lung disease features of claim 3, further comprising:
for each texture type of the predetermined list of texture types, calculating a total mass of each texture type within the region of interest by summing the mass of all voxels corresponding to each respective texture type within the region of interest; and
determining a percent contribution by mass for each texture type within the region of interest.

5. The method of characterizing lung disease features of claim 4, further comprising:
determining a composite score to characterize lung disease, the composite score comprising an algebraic sum of the percent contributions by mass of two or more texture types within the region of interest.

6. The method of characterizing lung disease features of claim 1, wherein the predetermined list of texture types includes two or more of the following texture types:
ground glass, reticulation, emphysema, air trapping on RV scans, honeycombing, consolidation, and normal lung tissue.

7. A method of characterizing lung disease features in terms of mass for each of a given classification type within a region of interest, the method comprising:
acquiring volumetric imaging data comprising a plurality of voxels;
defining a region of interest within the volumetric imaging data;
assigning a classification type to each voxel within the region of interest, the classification type for a given voxel selected from a predetermined list of classification types;
calculating a mass for each voxel based on its Hounsfield Units attenuation coefficients; and
calculating a total mass of a first classification type within the region of interest by summing the total mass of all voxels of the first classification type within the region of interest.

8. The method of characterizing lung disease features of claim 7, further comprising:
monitoring the total mass of the first classification type within the region of interest over time.

9. The method of characterizing lung disease features of claim 7, further comprising:
calculating a total mass of all voxels within the region of interest by summing the mass of all voxels within the region of interest; and
determining a percent contribution by mass of the first classification type within the region of interest.

10. The method of characterizing lung disease features of claim 9, further comprising:
for each classification type of the predetermined list of classification types, calculating a total mass of each classification type within the region of interest by summing the mass of all voxels corresponding to each respective classification type within the region of interest; and
determining a percent contribution by mass for each classification type within the region of interest.

11. The method of characterizing lung disease features of claim 10, further comprising:
determining a composite score to characterize lung disease, the composite score comprising an algebraic sum of the percent contributions by mass of two or more classification types within the region of interest.

12. The method of characterizing lung disease features of claim 7, wherein the predetermined list of classification types includes two or more of the following classification types:
normal, air trapping, or emphysema.

13. The method of characterizing lung disease features of claim 7, wherein the predetermined list of classification types comprises the following biomechanical properties:
normal elasticity and impaired elasticity.

14. A method of characterizing lung abnormalities in terms of a percentage by mass for each of a given texture type within a region of interest, the method comprising:
acquiring volumetric imaging data comprising a plurality of voxels;
defining a region of interest within the volumetric imaging data;
assigning a texture type to each voxel within the region of interest, the texture type for a given voxel selected from a predetermined list of texture types;
calculating a mass for each voxel within the region of interest based on its Hounsfield Units attenuation coefficients;
calculating a mass of each of the listed texture types within the region of interest by summing the mass of all voxels of each texture type within the region of interest;
calculating a total mass of all voxels comprising the region of interest by summing the total mass of all texture types within the region of interest; and
determining a percentage contribution by mass for at least one of the texture types within the region of interest by dividing the mass of the at least one texture type by the total mass of all voxels comprising the region of interest.
